(19) United States
(12) Patent Application Publication
(10) Pub. No.: US 2024/0041826 A1
(43) Pub. Date: Feb. 8, 2024

(54) Treatment for Diseases of Aging

(71) Applicant: Rory Powell Blake, Lancaster, SC (US)

(72) Inventor: Rory Powell Blake, Lancaster, SC (US)

(21) Appl. No.: 17/818,026

(22) Filed: Aug. 8, 2022

(51) Int. Cl. A61K 31/401; A61K 31/198; A23L 33/175; A23L 2/52; C12G 3/04; A23D 9/007; A23L 33/00

(52) U.S. Cl. CPC A61K 31/401; A61K 31/198; A23L 33/175; A23L 2/52; C12G 3/04; A23D 9/007; A23L 33/40; A23V 2002/00

(57) ABSTRACT

The human body's response to excessive gut bacterial growth is to deny the microbiome of two classes of amino acids. This halts exponential growth; however, the body is also denied their benefit. In the short term, prevention of explosive infection is accomplished. However, this triggers the necessity of removing the needed amino acids from bodily structure for the body to continue. This becomes the resulting long-term decline seen in human aging. This disclosure is methods to extend to the body the benefit of those missing classes of amino acids by providing them in a form less accessible to bacteria. The preferred embodiments claimed in this disclosure work in tandem to treat and to slow aging.

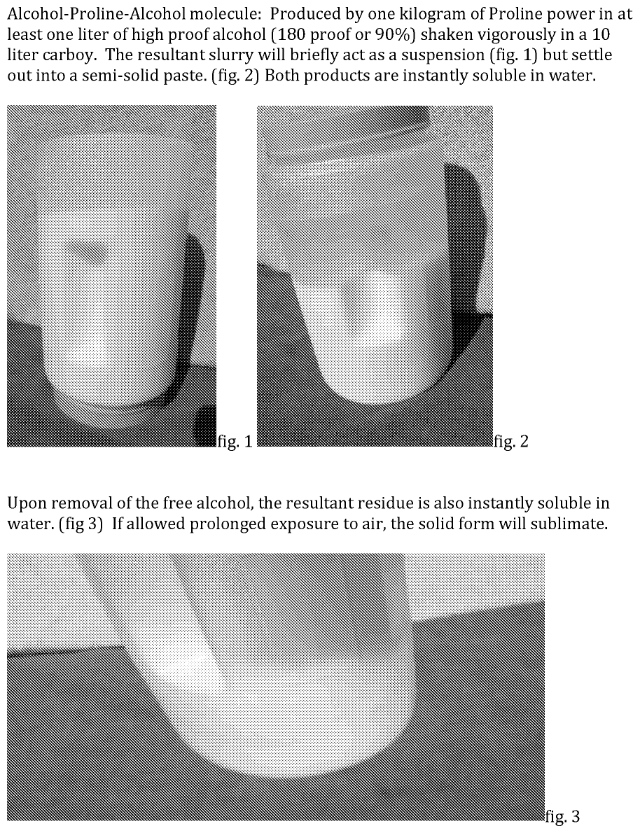

### CROSS-REFERENCE TO RELATED APPLICATIONS

| Publication | Publication | |
|---|---|---|
| number | date | Title |
| US693298781 | Aug. 23, 2005 | Chemical composition and method |
| | | for Enhanced Metabolism |
| U.S. Pat. No. | Oct. 10, 1972 | Amino acid food composition |
| 3,697,287A | | |
| U.S. Pat. No. | May 18, 1993 | Derivatives of I-proline, their |
| 5,212,168A | | preparation and their biological |
| | | uses |
| WO1995027408A1 | Oct. 19, 1995 | Sweetening agents containing |
| | | glycine, I-proline, or |
| | | I-hydroxyproline |
| US20080207733A1 | Aug. 28, 2008 | Composition for the Treatment of |
| | | Vitiligo |
| US20080317735A1 | Dec. 25, 2008 | Nutraceutical compositions with |
| | | biologically active ingredients |
| US20120282370A1 | Nov. 8, 2012 | Sparkling beverage and method of |
| | | producing same |
| CN101273775A | Oct. 1, 2008 | Cordyceps mycelium capsules and |
| | | producing process thereof |
| JP2001224334A | Aug. 21, 2001 | Beauty food containing amino acid |
| US656286981 | May 13, 2003 | Nutritional supplement for |
| | | increased energy and stamina |

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM (EFD-WEB)

Not Applicable

### STATEMENT REGARDING PRIOR DISCLOSURES BY THE INVENTOR OR A JOINT INVENTOR

| Publication | Publication | |
|---|---|---|
| number | date | Title |
| 61/741,990 | Aug. 1, 2012 | Biological basis for Adult Wellness |
| | | and Aging |
| 13/956,327 | Jul. 31, 2013 | Biological Basis of Adult Wellness |
| US 20160045473A1 | Sep. 23, 2017 | Biological Basis of Adult Wellness |

### BACKGROUND OF THE INVENTION

The field of the invention is treatments for aging related diseases. Nutrition, primarily amino acid nutrition is a secondary field of the invention. A secondary technical field is the support of physical human structure. This is accomplished by prevention of the alteration or subtraction of some of its components in order to provide for missing nutrition. Basically, the human body will steal from structure to continue during times of famine. The loss of physical structure is known to cause severe downstream affects. For example, a shortage of the imino acid, proline, results in it being removed from collagen lining the outside of small capillaries. This will eventually lead to weakness in the blood vessel wall and to the inevitable stroke.

Background Art includes many attempts to affect the genome to prevent aging and aging related diseases. However, this disclosure reveals that the DNA in the genome remains relatively intact during aging. The effect that aging appears to have on the body is epigenetic in nature. Epigenetic RNA cannot direct the repair and replacement of protein and peptides without the adequate supply of necessary amino acid nutrition. The present invention discloses the mechanism that the human body uses to control presumed parasitic infection in the gut. The body blocks absorption of two classes of amino acids to prevent gut parasites from obtaining this nutrition. There is a flaw in the body's nutritional parasite control mechanism. The body is also denied the benefit of the nutrition from these two classes of amino acids. This lack becomes the trigger of physical aging. The collapse of this nutritional control mechanism leads to aging relate disease and to the ultimate death of the organism.

### BRIEF SUMMARY OF THE INVENTION

That humans are more advanced than other animals cannot be totally explained by genome, as all fellow mammals have roughly the same number of genes. Human can have higher level function because we capture many more bacteria into our microbiome and convert those bacterial genes to do basic biological work. This scheme frees human DNA to code for higher level function. The method works but has a fatal flaw, aging decline. All higher animals incorporate some form of microbiome to help them with metabolism. Humans differ by incorporating hundreds more bacterial species into the microbiome. We also tolerate that we may undergo a long, slow decline.

All higher animals use an extra cellular DNA molecule known as a plasmid to control each individual bacteria living in the human gut. Over time, an individual bacteria will inevitably lose its plasmid. This is the fatal flaw. Without a plasmid to restrain growth, simple exponential growth rates can lead to an individual bacterium becoming tens of trillions of bacteria. Individually, each may only consume a small amount but taken together the unrestrained growth consumes an ever-larger share of the animal's protein intake. The human scheme adds an additional step to prolong life. Rather than simply dying at the conclusion of an internal starvation process like other animals, the human may first begin the process of a long physically decline.

Ultimately, the uncontrolled growth of gut bacteria leads to metabolic syndrome, other diseases of aging, and death. The human model squeezes out the last ounce of life before death.

The current inventor describes addition and replacement of plasmids in the human microbiome to control the overgrowth of bacteria. This application and disclosure is of the mechanism that the human body uses to mitigate overgrowth of bacteria, in situ. The body uses the epithelial lining of the intestine to break-down two classes of amino acids for energy. This serves to prevent parasites further-on in the intestine from obtaining sufficient nutrition to support explosive growth. The unfortunate side effect is that the human body is also denied benefit of this nutrition. It is, however, fortunate that both classes of amino acids targeted for destruction share the characteristic of being soluble in organic substances other than water. This becomes the principle behind the proposed solutions. This chemical characteristic could be used to both protect these amino acids from physical destruction and also hinder absorption by bacteria further-down in the gut. This disclosure involves several methods to deny nutrition to parasitic bacteria while also allowing that same nutrition to be absorbed for human body use.

### DETAILED DESCRIPTION OF THE INVENTION

The current inventor earlier describes a method of reducing aging related effects by the provision of additional specialized DNA molecules, or plasmids into the human gut. These plasmids were originally existent in each individual bacteria for the control of that individual bacterium. Once plasmid control is lost, bacteria exhibit unrestrained growth. Fortunately, bacteria readily accept plasmids. Plasmids are routinely shared between bacteria, even different species because of the useful information that they relay.

The present invention involves a work-around to the human body's normal response to excessive growth of gut bacteria. When sensing parasitic growth within the gut, the body begins to break down two classes of amino acids in the proximal epithelium of the intestine. Bacteria can produce their own amino acids, however, denying four of the most essential amino acids acts as a roadblock to exponential growth. The end result is, at best, a slowing of excess bacterial growth. The human body's system of dealing with parasitic growth is somewhat effective in the short term. Over extended periods, this normal method of bacterial control results in the catastrophic physical decline to the individual that is often seen in aging.

Human babies are born with copious amounts of plasmids. Human babies then acquire bacteria from the birth canal and elsewhere in infancy. When these newly captured bacteria begin to multiply, the existent plasmids become incorporated into each of the bacteria. These plasmids along with bacterial DNA will then direct the function of the bacteria. Plasmid control continues as its further replicates within all bacteria in the microbiome.

Over time, and under specific circumstances some bacteria in the microbiome can lose their plasmids. Plasmids, among other tasks, restrain replication. The loss of plasmid control can lead to uncontrolled growth of bacteria, even as it remains a part of the microbiome. The immune system continues to recognize this as friend, even though, it has now become parasitic and is foe.

Experimental evidence shows that a lack of plasmids will lead to explosive and exponential growth of gut bacteria. The explosive growth of “rogue bacteria” has been shown to be a major cause of parasitic drag on the human body. The experimental addition of plasmids seems to reestablish control of rogue bacteria within the microbiome. This appears as the best way to stop exponential bacterial growth without having to tolerate physical decline.

The body's normal method of denying specific nutrition to bacteria to slow explosive growth is effective. However, it takes quite a toll on the body's structure as can be observed in late-stage aging. This is because the body's method of break-down of specific nutrition also denies benefit of that nutrition to the human body and begins the body having to steal from structure to continue forward. This disclosure is of a method of replacement of the specific nutrition in a form unavailable to bacteria.

Live-bearing fishes of the Goodeid family were chosen as subjects for these experiments because they are intelligent and highly adaptable. Goodeid's can occupy a similar number of diverse ecological niches as do humans. These fish seem to occupy the same types of biological and social niche as do humans, only in water. Findings within study of these animals should apply directly to humans within these categories of nutrition and digestion. An additional reason for choosing fish as subjects is that every important biological perimeter can be controlled within a water tank. Humans do not notice the excessive growth of gut bacteria because to them is simply disappears in the stool. The fish tank environment allows for the capture of, and accounting for, all excess bacterial growth.

As in humans, metabolic syndrome can be provoked in fish by overfeeding. This results in the ultimate death of the fish by the apparent overgrowth of what was once normal gut bacteria. Since fish do not have the acidic stomach of humans, additional bacteria may easily join the bacteria already lining the GI tract. The propensity for excessive growth of bacteria may be behind the warning on fish food packages that uneaten food should be promptly removed from the tank. The excess food may be easily self-contaminated or spoiled by the unchecked growth of normal bacteria. This would be evidence that uncontrolled exponential increase of gut bacteria may be behind the phenomenon of metabolic syndrome in humans, as well.

Additionally, in adult fish, aging and the ultimate death of the organism can be rapidly speeded. This death can be triggered by the addition of “old” bacteria to their water. In fish, the introduction of the aged version of their normal gut bacteria to their water will lead to death within days. The fish stop eating, become docile and cease all normal activity. Death from the addition of “old” bacteria will only occur in adult fish. Factors resulting in “old” bacteria are uncontrolled growth of gut bacteria in situ, as well as, the addition of normal bacteria cultured separately from the fish. Both forms of “old” bacteria will lead to death in adult fish. Fish aged experimentally or that occurring naturally do not experience the extreme aging decline seen in humans.

Goodeid fishes, like humans, are born without gut bacteria. Transferred to conditioned water that is lacking bacteria, the baby fish soon stiffen, become rigid and die. Interestingly, the motionless fry may be transferred to tanks inhabited by a plurality of very young fish and recover. Further, this recovery will often occur if the motionless fry be placed into the tanks of Goodeids which do not share the identical bacteria with the others of their species. This recovery will not occur without the water predominately being shared by active fry. The only factor that can account for this surprising recovery is the presence of many young.

This confirms that fry of Goodeid fish must acquire a microbiome to survive. Further, it appears that when unable to train their own bacteria the fry can acquire bacteria conditioned by other young fish. In general, Goodeid species survive in the presence of diverse types of bacteria in their water. They adapt well to new bodies of water. Adults seem to carry their own gut bacteria while the newly born young acquire a new microbiome. The new microbiome may include a combination of the parent's bacteria and the bacteria present and already adapted to the water in the new environment. The complete data suggests that while fry may be born without bacteria, they are born with plasmids that allow control of whatever bacteria may be encountered or is present in the environment. Human infants are born with plasmids derived from placental bacteria. Goodeids have a similar organ to the human placenta that provides plasmids.

Serious inquiry into the microbiome cannot be done without the ability to culture a complete microbiome. This has now been accomplished. This allows for experiments involving substitution of the intact microbiome. This tool is an “internal tank filter” that was developed to allow growth of both aerobic and anaerobic bacteria. The cultured intact microbiome can be kept alive for extended periods of time. While over time this may be toxic to adult fish, the above microbiome soup will allow for the development of new fry. This tool allowed for the creation of entirely new species of fish via the transfer of newly born fry to tanks containing a different microbiome from their parents.

The gut has long been referred to as the center of emotion and response in humans. This applies to fish, as well as. It would be fair to assume that gut bacteria play a role in the production of neurotransmitters. Fry that have not yet developed a microbiome become paralyzed when the initial supply of neurotransmitters becomes depleted. The “Old” bacteria placed in the water will produce hypnotic molecules that seem very attractive to fish and also seem responsible for symptoms of old age such as lethargy and dizziness. These symptoms seem to occur in adult fish that are exposed to “old” bacteria.

The data suggests that normal plasmid replication within bacteria cannot always be expected to keep pace with exponential bacterial replication. This is the flaw in using bacteria to produce basic building blocks for higher lifeforms. This is true “over time” and especially with some specific bacteria species that exhibit a characteristic exponential growth. Pepto Streptococcus is a common human gut bacterium. It has been known to quadruple in as few as three hours when exposed to carbon monoxide. Since carbon monoxide is a common contaminant of carbon dioxide produced in the production of alcohol, Pepto Streptococcus has been implicated in “beer belly” or metabolic syndrome. There can be little wonder that humans can harbor literally trillions of gut bacteria.

The explosive growth of “Rogue Bacteria” unrestrained by plasmids may indeed trigger aging due to competition for resource within the organism. In defense of the previous researchers, it is noted that bacteria are actually capable of producing all of their own amino acids.

Fish do not have a “traditional acid stomach” used to digest food intake, therefore, all intake either remains in the gut as microbiome or broken down for nutrition. This may explain why fish may eat their young . . . as to obtain new plasmids. Twenty-pound bass are as much as four times as old as the average adult bass. They can grow this old by consuming a multitude of fry. The microbiome contained in each of the young fish successfully regenerates the gut bacteria of the older fish.

The human body's method of breaking down specific amino acids is effective in denying specific nutrition to explosively growing gut bacteria but also results in the generalized structural decline characteristic of human aging. The obvious solution would be to formulate a way to deny the nutrition to bacteria and to enhance the absorption into human cells.

The disclosure involves placing these specific amino acids into the diet in a form that can be absorbed by the human body yet the gut bacteria cannot absorb.

The 122-year lifespan achieved by Jeanne Calment is illustrative of this process. Madam Calment enjoyed a bottle of port wine each night with a leisurely dinner for decades and decades. The port wine has enough alcohol to bind with the only alcohol soluble amino acid, proline, while both were in the stomach. This allowed for proline to be absorbed through the stomach rather than traveling to the intestine where it would be broken down in the body's effort to prevent gut bacteria from gaining access. Combining proline with alcohol was successful in increasing longevity. Over time, this small amount of proline absorbed made a huge difference in the longevity of Madam Calment.

Aging researcher David Sinclair studied the case of Jeanne Calment and used it as the basis for his claim that red wine with meals is good for longevity. He further named an obscure component of red wine; however, the alcohol has been tested and been confirmed as the true active ingredient.

Current embodiments of the alcohol-proline-alcohol molecule are many times the concentration that which Madame Calment obtained in her nightly meal and has successfully undergone clinical trials since 2012. The rest of her nightly meal is unrecorded; however, the Mediterranean diet is widely understood as being healthy and important to longevity. The Mediterranean diet is also notable for the amount of olive oil that it contains. Branch chain amino acids combined with olive and other oils has had only limited testing, however, branch chain amino acids are known as one of the two groups of amino acids that are broken down in the intestine to prevent gut bacteria from obtaining it. Branch chain amino acids are generally appreciated as important and are known to be lacking in the average healthy diet.

The human circulatory system, pumped by the heart, moves oxygen and glucose around the body. The human lymph system moves fatty acids and amino acids from the intestine to the 400 lymph nodes. As the lymph does not have a pump, it is moved around the body solely by muscle movements. We were designed as hunter gatherers. Therefore, muscle movement should remain sufficient to carry nutrients around the body. This is why walking is so important to health and longevity.

There are 400 lymph nodes that are centers for epigenetic work around the body. For RNA and a ribosome to produce protein would require twenty specific amino acid carriers, one for each amino acid. This allows need for eight-thousand individual amino acid carriers to direct each amino acid to the area of intended need. Humans likely have so many carriers as to prioritize importance of need during famine. It is also likely that higher level carriers are permissioned to steal needed amino acids from structure. This ability to remove needed amino acids would explain much of the damage observed in non-essential or less essential body parts during extended aging.

The current disclosure is of an alcohol-proline-alcohol molecule that has ability to absorb through the stomach rather than traveling to the intestine where it would normally be destroyed were it a molecule of proline, alone. This compound is formed when purified proline powder is with a high percentage of alcohol and with agitation. Also disclosed are molecules of the branched chain amino acids, lucine, valine and isoleucine combined with vegetable oil. The branched side-chains of these amino acids are extremely soluble in oil. This combination if formed by purified valine, isoleucine and lucine being stirred into olive or other oil under very low heat. The combination of these branched chain amino acids in oil will resist incorporation into bacteria, as well as, have enhanced absorption by human amino acid carrier molecules of the lymph system.

The human body's initial response to the dangerous overgrowth of bacteria is to block the nutrition of two classes of amino acids from reaching the bacteria in the colon. These two classes are imino acids and branch chain amino acids. The replacement of these becomes essential to halting aging.

In humans, the onset of aging is at once more gradual and ultimately more extreme because of a further epigenic cause. This epigenetic failure is due to a lack of specific amino acid nutrition. This lack is triggered by the voluntarily break-down of amino acids in the body to keep “rogue bacteria” from obtaining them.

In aging, the genome remains intact; however, RNA cannot code protein without having available all amino acids necessary to build that particular protein. Deficit in protein repair, as well as, a decline in the peptides responsible for the regulation of bodily function can be observed in human aging. Provision of missing amino acid nutrition goes a long way towards repair of aging damage.

There are twenty-four data points that link aging to the amino acid proline and to the plasmids of gut bacteria.
1. It is in the literature that proline is the exclusive energy source for every parasite. It is also the energy source for spermatozoa. The test for semen is actually a test for proline.
2. Studies in the 1960′s showed a 30% lower lifespan in sexually active male Flatworms. Males of most animal species have reduced lifespan compared to females. Potentially the loss of proline may be a factor.
3. It is in the literature that the human body breaks down proline in the epithelium of the intestine for an unspecified reason, long thought to be for energy production purposes. It is my contention that this is a response to parasitic infection in the gut.
4. Proline was once considered a non-essential amino acid. Now it is considered as conditionally essential especially in the elderly by many sources.
5. Proline is the amino acid responsible for “springiness” in protein. Lack of proline causes deficiencies in collagen. This results in joint weakness and is also responsible for saggy skin in older humans.
6. Proline is the only amino acid that is more soluble in alcohol than it is in water. This could be a factor behind longevity increases in moderate drinkers. Moderate drinking is considered alcohol with meals.
7. Proline was decided as non-essential in the 1930's. My contention is that the body is removing it from structure rather than at assembling it de-novo. There was no way for 1930's science to detect the difference.
8. The human lymph system is more complicated than that of other animals. The human lymph system is responsible for delivering amino acids from the gut to 400 lymph nodes. This is likely to allow for a hierarchy of need that is useful in famine. Twenty amino acids directed to 400 lymph nodes would be unique 8,000 carriers. Some carriers with superior function could remove amino acids from structure to provide for more important need. This may be how humans can last 30-60 days without food. This may also be why aging appears much like starvation in older humans.
9. In long-term care, parenteral nutrition amino acid supplementation often appears helpful in treating physical decline in late-stage aging. Per my clinical observations, these patients relapse quickly when treatment is stopped.
10. The gut bacteria present in every nursing home patient is dominated by one species of bacteria. This suggests an overgrowth of one bacterial species potentially overwhelming all of the rest. I cannot explain how other clinicians have missed this.
11. Serotonin and other neurotransmitter production is associated with the gut. The gut has often been called a second brain or the center of emotion. Overgrowth of parasitic bacteria overcrowding other bacteria in the gut may affect cognition and alertness. This lack of neurotransmitter production could be a reason for the mental decline of aging.
12. No animal is born with a microbiome. Humans are born with a gut full of DNA tokens known as plasmids. These are produced by placental bacteria. Placental bacteria were so unexpected that it now has its own Wikipedia page.
13. Gut bacteria appear to be organized into the microbiome via these small DNA tokens called plasmids. Any bacteria that replicate in the gut of the newborn receives a token. Bacteria losing their plasmid would allow for excessive and uncontrolled growth of that bacterium and, of course, all subsequent daughter cells.
14. The bacteria species Pepto Streptococcus has been shown to quadruple in as little as three hours. Although capable, during periods of this explosive growth the bacteria does not have time to produce its own supply of amino acids. They consume the protein groceries taken in by the human host.
15. Gut bacteria may not initially appear as parasitic because many of the resulting daughter cells leave the body in the stool. This is wholly unlike the giant tapeworm whose ample growth exposes it as a parasite. My experiments with fish in a contained system allows an ability to account for excessive bacterial growth which remains visible within the confines of the fish tank.
16. Jeanne Calment, the longest living human, spent every evening for one hundred years partaking of a long, leisurely dinner which involved consuming an entire bottle of port wine. This led to many stating that wine is good for longevity. My contention is that the alcohol in the port wine combined with proline from the partially digested protein. This allowed for proline to be absorbed through the stomach along with the alcohol and keeping the proline from the lower gut.
17. Walking is possibly seen as good for longevity due to its effects on the lymph system. Humans were designed as hunter gatherers, therefore walking enhances lymph circulation. The circulatory system moves oxygen and glucose due to the beating of the heart. Amino and fatty acids are moved from the gut to the lymph nodes via muscle movement. Without the movement of nutrition from the gut, harmful gut bacteria consume a multiplicity of the amino acids—while the fatty acids are deposited near to where they are absorbed. This accounts for the growth of the middle areas and obesity, while at the same time, the individual may be starving-to-death.
18. Moderate daily use of Coffee and Tea are shown to be useful for increased longevity. This may be due to the ability of caffeine to enhance regular bowel function and eliminate excess bacteria during times of exponential bacterial growth.
19. Aging has been shown to be epigenetic in nature. Epigenetic function involves RNA being used as a template for building protein. Even perfect RNA cannot be capable of building protein without having all necessary amino acids needed to build that protein.
20. In aging, Hormonal decline occurs in a gradual manner. Were it DNA damage, the drop in hormone production would be catastrophic. Lack of, or irregular supply of specific amino acids would be sufficient to cause this gradual decline.
21. The first diseases of aging are diabetes and hypertension. Both of these are caused by control peptide deficiencies, incretins and renin/angiotensin.
22. If proline is an essential amino acid and broken down in the epithelial cells of the intestine, when ingested, it would not be available for protein without being robbed from existing structure. Visible decline of collagen would be a sign of this phenomenon.
23. Early clinical trials of proline alcohol were successful and included unexpected results. Trials were halted due to herpes outbreaks in those who had cold sores as younger individuals. Herpes is called “cold sores” or “fever blisters” due to it hiding behind the blood brain barrier until it determines that the immune system is busy elsewhere. The amino acid, Lysine, is known to prevent outbreaks. High levels of proline may be a sign to the virus that the immune system is fighting an infection, elsewhere. The body may break down collagen to obtain proline as the normal method. It is my contention that the body breaks down collagen in emergency situations as it cannot readily produce it.
24. Alzheimer's disease is associated with aging and the production of plaque deposits in the brain. The individuals described above had not experienced “fever blisters” in twenty years, or more. The amyloid plaque of Alzheimer's has been ascribed to immune function. There is possibility that the immune product is in response to herpes or other virus living in the brain. This shows the effect proline has on one of aging's most disturbing signs and symptoms.

1: A water soluble slurry of Alcohol-Proline-Alcohol.

2: The product of claim 1 diluted with water and intended as a liquid beverage.

3: The product of claim 1 delivered in an encapsulated form.

4: The product of claim 1 diluted and combined with flavored water and delivered in the form of an oral beverage.

5: The product of claim 2 combined with sufficient carbonation and/or additional water or carbonated water and further intended for use as an oral soft drink product.

6: The product of claim 4 with the addition of sufficient water and/or alcohol to be classified as an alcoholic beverage.

7: The product of claim 5 combined with sufficient alcohol to be classified as an alcoholic beverage.

8: The product of claim 5 combined with sufficient amino acids, electrolytes and nutrients and intended as an oral therapeutic.

9: The product of the amino acid Luecine combined with an edible oil.

10: The product of the amino acid Isoleucine combined with an edible oil.

11: The product of the amino acid Valine combined in an oleaginous suppository form.

12: The product of the amino acid Leucine combined in an oleaginous suppository form.

13: The product of the amino acid Isoleucine combined in an oleaginous suppository form.

14: The product of the amino acids Valine and Isoleucine in an oleaginous suppository form.

15: The product of claim 14 where Leucine is substituted for Isoleucine.

16: The product of claim 14 where Leucine is substituted for Valine.

17: The product of claim 14 where Leucine is added to Valine and Isoleucine.

18: The product of claim 17 intended for vaginal use.

19: The product of claim 17 combined with the product of claim 1.

20: The product of claim 17 combined with proline powder.
